(19) United States
(12) Patent Application Publication
(10) Pub. No.: US 2026/0076690 A1
(43) Pub. Date: Mar. 19, 2026

(54) MEDICAL DEVICE

(71) Applicant: ASAHI INTECC CO., LTD., Seto-shi, (JP)

(72) Inventors: Shintaro KAWAI, Seto-shi (JP); Erina MATSUOKA, Seto-shi (JP); Yamato HIRATA, Seto-shi (JP)

(73) Assignee: ASAHI INTECC CO.,LTD., Seto-shi. (JP)

(21) Appl. No.: 19/328,993

(22) Filed: Sep. 15, 2025

(30) Foreign Application Priority Data
Sep. 18, 2024 (JP) 2024-160688

(51) Int. Cl. A61B 17/22; A61B 17/00; A61M 25/09

(52) U.S. Cl. CPC A61B 17/22; A61B 17/00234; A61M 25/09; A61B 2017/00238; A61B 2017/00292; A61B 2017/22042; A61B 2017/22094; A61M 2025/09083; A61M 2025/09175

(57) ABSTRACT

A medical device includes an elongated main body part and a leading portion connected to a distal end of the main body part and enters a lesion. When viewed from a first direction orthogonal to an axial direction of the main body part, an outer edge of the leading portion satisfies at least one of (1) a first condition that the outer edge has a first end portion located at a distal end of the leading portion and a second end portion located on a side opposite to the first end portion across a center axis of the main body part in a second direction orthogonal to both an axial direction of the main body part and the first direction, the second end portion being convex toward the distal end side, and (2) a second condition that the outer edge has a straight portion extending in the second direction.

### CROSS REFERENCE TO RELATED APPLICATIONS

This is a Paris Convention application based on JP 2024-160688 filed on Sep. 18, 2024. The disclosure of the prior application is hereby incorporated by reference in its entirety.

### TECHNICAL FIELD

A technology as disclosed herein relates to a medical device.

### BACKGROUND ART

A known medical device includes a loop at a distal end portion thereof (for example, see Patent Document 1).

### Citation List[0003]

Patent Document 1: Japanese Unexamined Patent Publication No. 2010-502378

### SUMMARY

### Technical Problem

There is room for improvement in passability of a medical device through a lesion.

The present specification discloses a technology that can solve the above-described problem.

### Solution to Problem

The technology as disclosed herein can be achieved, for example, as the following aspect.

A medical device as disclosed herein includes an elongated main body part, and a leading portion that is connected to a distal end of the main body part and enters a lesion. When viewed from a first direction orthogonal to an axial direction of the main body part, an outer edge of the leading portion satisfies at least one of (1) a first condition that the outer edge has a first end portion located at a distal end of the leading portion and a second end portion located on a side opposite to the first end portion across a center axis of the main body part in a second direction orthogonal to both the axial direction of the main body part and the first direction, the second end portion being convex toward a distal end side, and (2) a second condition that the outer edge has a straight portion extending in the second direction.

### BRIEF DESCRIPTION OF DRAWINGS

FIG. 1 is an explanatory view schematically illustrating a configuration of a guide wire according to a first embodiment.

FIG. 2 is an explanatory view schematically illustrating a configuration of the guide wire according to the first embodiment.

FIG. 3 is an explanatory view schematically illustrating a configuration of the guide wire according to the first embodiment.

FIG. 4 is an explanatory view schematically illustrating a configuration of the guide wire according to the first embodiment.

FIG. 5 is a flowchart illustrating an example of a treatment method using the guide wire.

FIG. 6 is an explanatory view illustrating an example of the treatment method using the guide wire.

FIG. 7 is an explanatory view illustrating an example of the treatment method using the guide wire.

FIG. 8 is an explanatory view illustrating an example of the treatment method using the guide wire.

FIG. 9 is an explanatory view schematically illustrating a configuration of a guide wire according to a second embodiment.

FIG. 10 is an explanatory view schematically illustrating a configuration of a guide wire according to a third embodiment.

FIG. 11 is an explanatory view schematically illustrating a configuration of a guide wire according to a fourth embodiment.

FIG. 12 is an explanatory view schematically illustrating a configuration of a guide wire according to a fifth embodiment.

FIG. 13 is an explanatory view schematically illustrating a configuration of a guide wire according to a sixth embodiment.

FIG. 14 is an explanatory view schematically illustrating a configuration of a guide wire according to a seventh embodiment.

### DETAILED DESCRIPTION

### A. First Embodiment

### Basic Configuration of Guide Wire 100

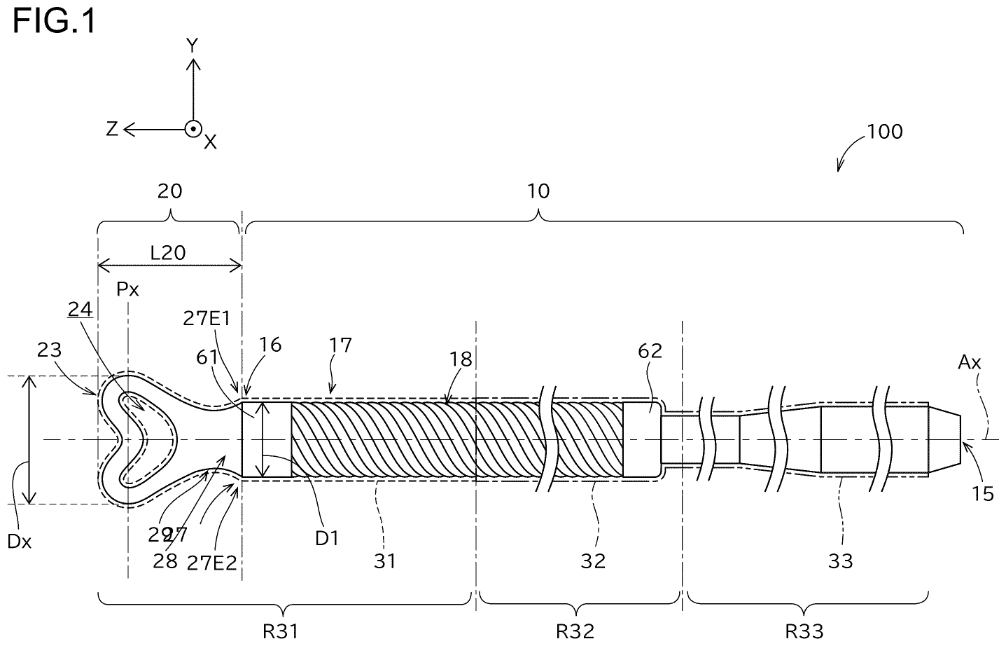

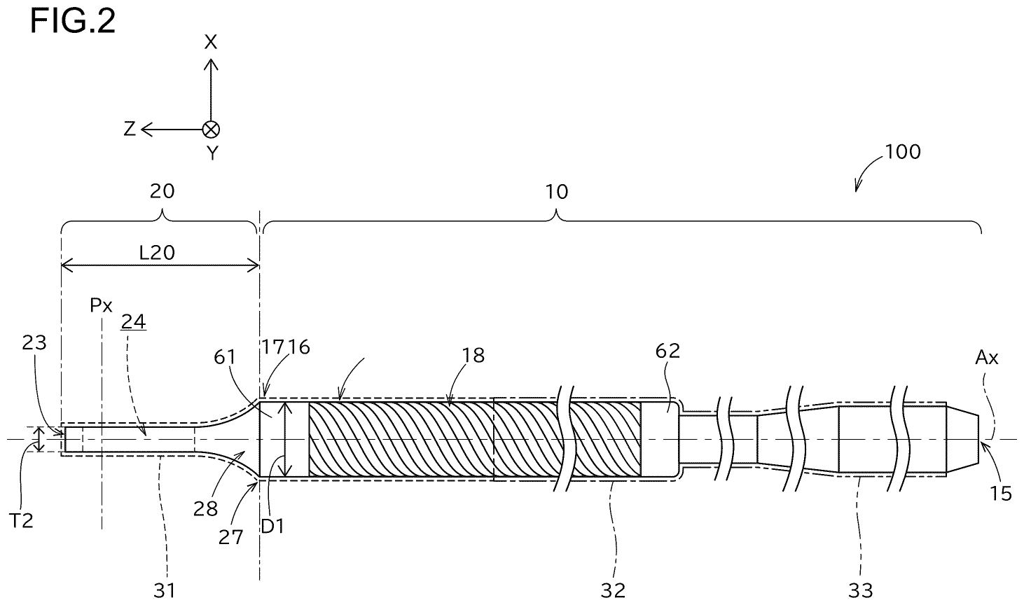

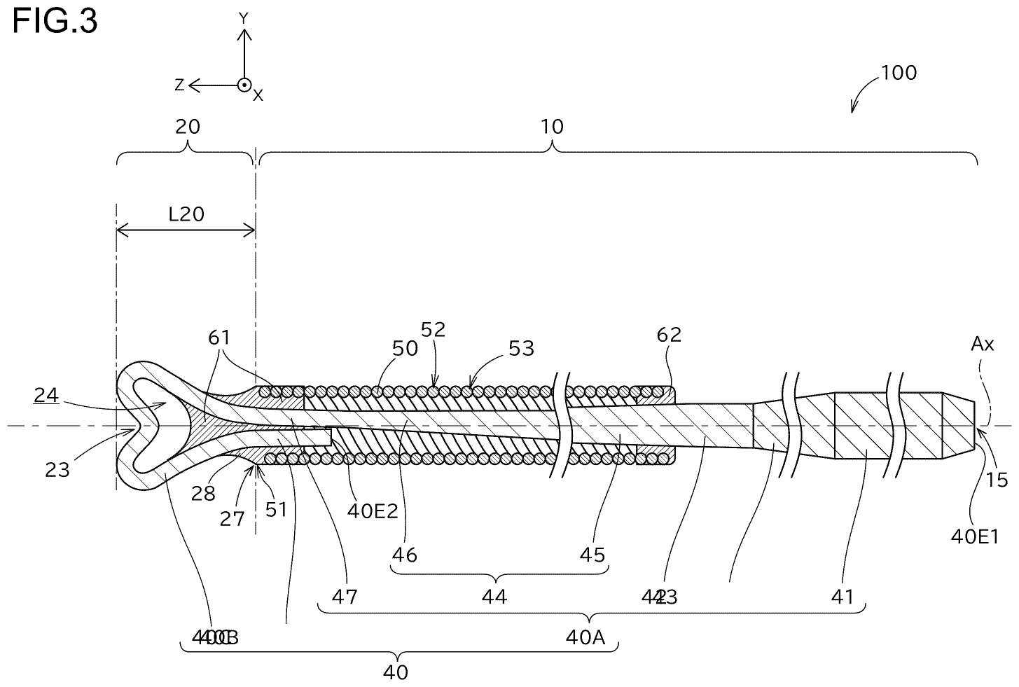

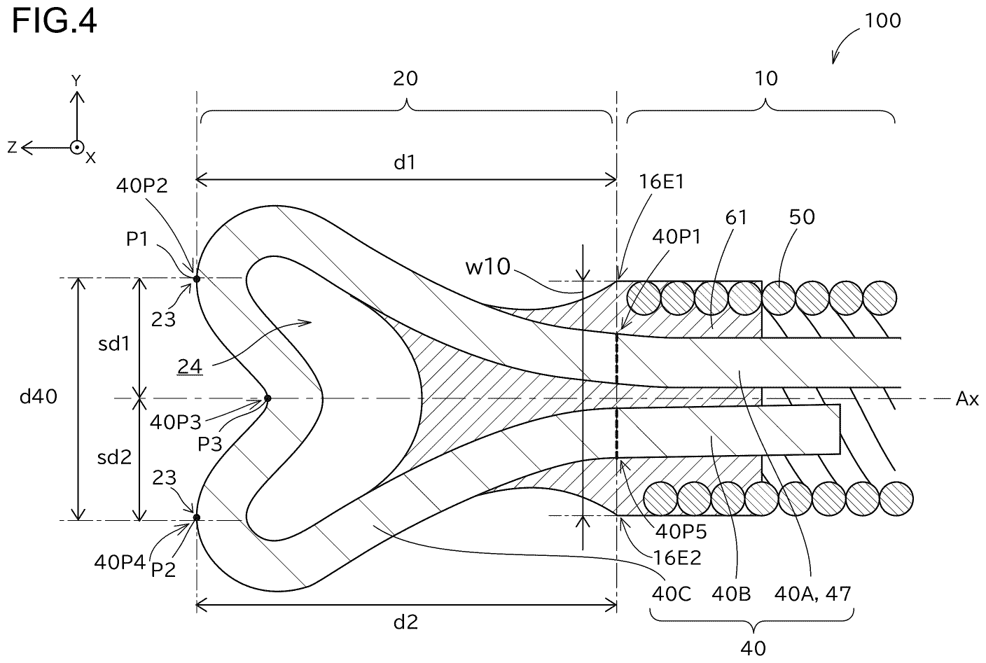

FIGS. 1 to 4 are explanatory views schematically illustrating a configuration of a guide wire 100 according to a first embodiment. In each drawing, XYZ axes orthogonal to each other for specifying a direction are illustrated. FIG. 1 illustrates an external appearance of the guide wire 100 as viewed in an X-axis direction. FIG. 2 illustrates an external appearance of the guide wire 100 as viewed in a Y-axis direction. FIG. 3 illustrates a YZ longitudinal section of the guide wire 100. FIG. 4 illustrates a YZ longitudinal section of a distal end portion of the guide wire 100. In the guide wire 100, the positive side of the Z-axis is a distal end side (far side) to be inserted into a body. In the guide wire 100, the negative side of the Z-axis is a proximal end side (near side) to be operated by a professional. In each drawing, a part of the guide wire 100 may be omitted. FIGS. 1 to 3 illustrate a state where the guide wire 100 is in a linear shape parallel to the Z-axis. The guide wire 100 is flexible enough to be curved. These points are also the same in the subsequent drawings. The X-axis direction is an example of a first direction. The Y-axis direction is an example of a second direction.

In the present specification, regarding the guide wire 100 and each component thereof, an end on the distal end side is referred to as a “distal end”, the distal end and the vicinity thereof are referred to as a “distal end portion”, an end on the proximal end side is referred to as a “proximal end”, and the proximal end and the vicinity thereof are referred to as a “proximal end portion”. A transverse section of the guide wire 100 and each component thereof means a cross section orthogonal to a longitudinal direction. A longitudinal section of the guide wire 100 and each component thereof means a cross section parallel to a center axis in the longitudinal direction. In the guide wire 100 and each component thereof, a direction orthogonal to the longitudinal direction is referred to as a “radial direction”. An outer diameter of the guide wire 100 and each component thereof means a width along the radial direction. In the present specification, regarding the guide wire 100 and each component thereof, a length along the Y-axis direction may be particularly referred to as a “width”, and a length along the X-axis direction may be particularly referred to as a “thickness”.

The guide wire 100 is an elongated medical device to be inserted into a living body lumen such as a blood vessel. An entire length of the guide wire 100 is, for example, not less than 1000 mm and not more than 3000 mm. The guide wire 100 is an example of the medical device.

The guide wire 100 includes a main body part 10 and a leading portion 20.

The main body part 10 is an elongated portion extending along a center axis Ax. In the present embodiment, the center axis Ax of the main body part 10 coincides with the center axis of the guide wire 100. A proximal end 15 of the main body part 10 coincides with the proximal end of the guide wire 100. A spiral groove 18 is formed on an outer peripheral surface 17 of a distal end portion of the main body part 10.

The leading portion 20 is connected to a distal end 16 of the main body part 10. A proximal end 27 of the leading portion 20 is connected to the distal end 16 of the main body part 10. The leading portion 20 can be expressed as a guiding portion, a drilling portion, a fracturing portion, a peeling portion, an advancing portion, a peeler, a shaver, or the like. A distal end 23 of the leading portion 20 coincides with the distal end of the guide wire 100. The leading portion 20 has a loop shape surrounding a through-hole 24 extending in the X-axis direction. A surface of the leading portion 20 may have an edge or may not have an edge. The edge is a boundary (ridge line) between two surfaces. The leading portion 20 enters a lesion while rotating around the center axis Ax. The entry of the leading portion 20 into the lesion can be expressed as crossing/passing through the lesion, drilling the lesion, fracturing the lesion, peeling the lesion, pushing through the lesion, diving into the lesion, advancing into the lesion, and the like. A length L20 of the leading portion 20 along the center axis Ax is, for example, not less than 0.2 mm and not more than 2.0 mm. The length L20 of the leading portion 20 may be not less than 0.3 mm and not more than 1.5 mm, or may be not less than 0.4 mm and not more than 1.0 mm. When viewed in the X-axis direction, an end portion of the proximal end 27 of the leading portion 20 on the positive side of the Y-axis is referred to as a first end portion 27E1, and an end portion of the proximal end 27 of the leading portion 20 on the negative side of the Y-axis is referred to as a second end portion 27E2.

A maximum outer diameter Dx of the leading portion 20 is larger than a thickness T2 of the leading portion 20 at a maximum outer diameter position Px. That is, the leading portion 20 has a flat shape as a whole. The thickness T2 of the leading portion 20 at the maximum outer diameter position Px is smaller than a maximum outer diameter D1 of the distal end 16 of the main body part 10. The thickness T2 of the leading portion 20 at the maximum outer diameter position Px is, for example, not less than 0.02 mm and not more than 0.3 mm. The thickness T2 of the leading portion 20 at the maximum outer diameter position Px may be not less than 0.04 mm and not more than 0.2 mm, or may be not less than 0.06 mm and not more than 0.1 mm. The maximum outer diameter Dx of the leading portion 20 is, for example, not less than 1.2 times the thickness T2 of the leading portion 20 at the maximum outer diameter position Px. The maximum outer diameter Dx of the leading portion 20 may be not less than 1.5 times or not less than 1.8 times the thickness T2 of the leading portion 20 at the maximum outer diameter position Px.

The maximum outer diameter Dx of the leading portion 20 is measured as follows. A measurer observes the guide wire 100 from a side thereof. The side is the Y-axis direction in the present embodiment. The measurer searches for an angle at which a portion on the near side and a portion on the far side of the leading portion 20 overlap each other and the portion on the far side cannot be seen. For example, when both of the following two conditions are satisfied, the measurer searches for an angle at which a portion between the second end portion 27E2 and the distal end 23 cannot be seen. The first condition is that a portion between the first end portion 27E1 and the distal end 23 is located on the near side. The second condition is that the portion between the second end portion 27E2 and the distal end 23 is located on the far side. The invisibility of the portion between the second end portion 27E2 and the distal end 23 is caused by an overlap between the portion between the first end portion 27E1 and the distal end 23 and the portion between the second end portion 27E2 and the distal end 23. Next, the measurer photographs the guide wire 100 by using a microscope along a viewpoint rotated by 90 degrees around the center axis Ax from the viewpoint at this time. The measurer sets a photographing magnification of the microscope to not less than 200 times. The measurer measures the outer diameter of the leading portion 20 at three measurement positions where the leading portion 20 is considered to have the maximum outer diameter Dx on the captured image. Specifically, at each measurement position, the measurer draws a pair of straight lines in parallel to each other, which pass through a pair of end portions in an outer diameter direction of the leading portion 20 and are orthogonal to the outer diameter direction, and measures an interval between the pair of straight lines. The measurer adopts a maximum value of measurement results at the three measurement positions as the maximum outer diameter Dx of the leading portion 20.

### Treatment Method Using Guide Wire 100

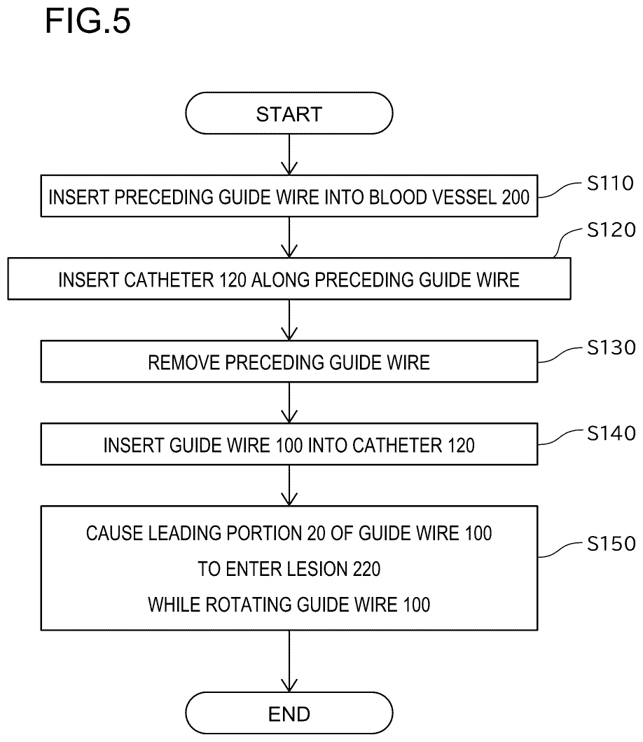

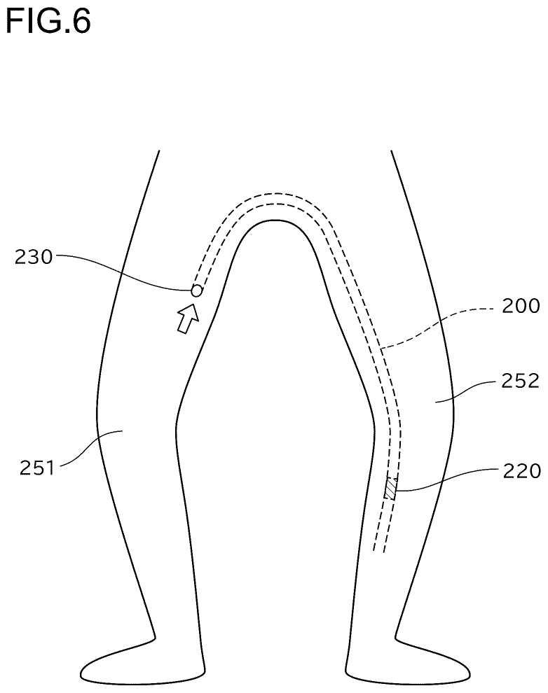

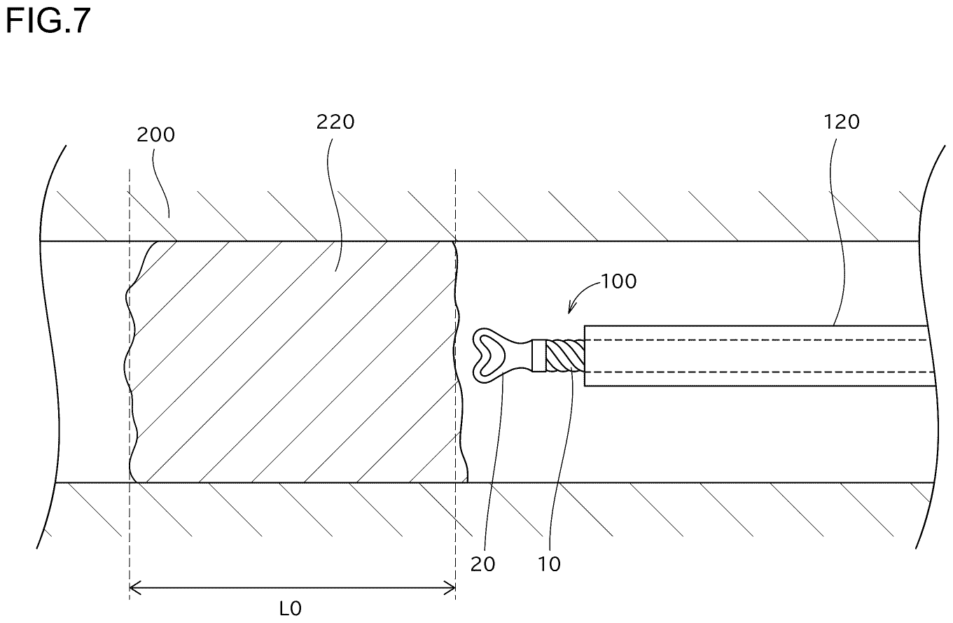

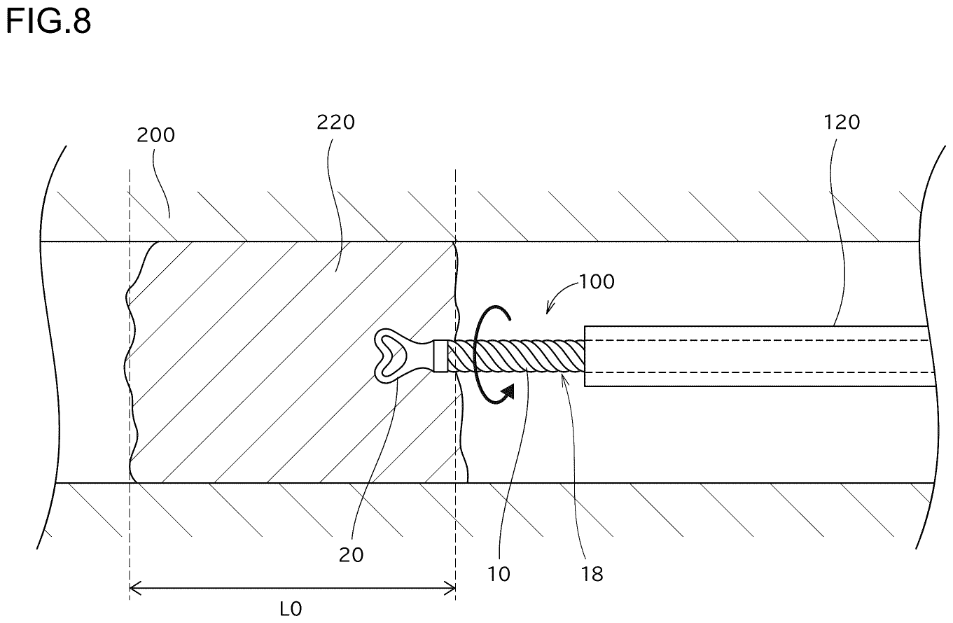

FIG. 5 is a flowchart illustrating an example of a treatment method using the guide wire 100. FIGS. 6 to 8 are explanatory views illustrating an example of the treatment method using the guide wire 100. As illustrated in FIGS. 7 and 8, in the treatment method using the guide wire 100, a professional causes the leading portion 20 of the guide wire 100 to enter a lesion 220 in a blood vessel 200. The lesion 220 is, for example, a highly calcified lesion. The lesion 220 is, for example, a chronic completely occlusion lesion. A length L0 of the lesion 220 along the extending direction of the blood vessel 200 is, for example, not less than 100 mm and not more than 500 mm. The length of the lesion 220 may be not less than 150 mm and not more than 450 mm, or may be not less than 200 mm and not more than 400 mm.

As illustrated in FIG. 6, the lesion 220 to be treated is located, for example, in the blood vessel 200 of the lower limb of a human. FIG. 6 illustrates a lesion 220 that has occurred in a below-knee region. In this treatment method, for example, a technology called crossover is used in which the blood vessel 200 is accessed from a groin of an opposite leg 251, which is the leg opposite to a target leg 252 in which the lesion 220 is located, and the lesion 220 in the target leg 252 is approached. A method of approaching the lesion 220 that has occurred in the below-knee region by inserting the guide wire 100 into the blood vessel 200 may be a method other than the crossover. The approach method may be an antegrade approach in which the guide wire 100 is inserted through the blood vessel 200 in a groin of the target leg 252 in which the lesion 220 is located, and the guide wire 100 is advanced along the flow of the blood stream. The approach method may be an antegrade approach in which the guide wire 100 is inserted through the blood vessel 200 in the arm and the guide wire 100 is advanced along the flow of the blood stream. The approach method may be a retrograde approach in which the guide wire 100 is inserted through either the blood vessel 200 in the ankle or the blood vessel 200 in the dorsum of the foot, of the target leg 252 in which the lesion 220 is located, and the guide wire 100 is advanced against the flow of the blood stream. The position at which the guide wire 100 is inserted into the blood vessel 200 is not limited to the above-described position. When the guide wire 100 is inserted through the blood vessel 200 in the arm, the professional can select the blood vessel 200 in the wrist as a position at which the guide wire 100 is inserted into the blood vessel 200. When the guide wire 100 is inserted through the blood vessel 200 in the leg, the professional can select a superficial femoral artery or a popliteal artery as the position at which the guide wire 100 is inserted into the blood vessel 200. The guide wire 100 is not limited to the treatment of the lesion 220 that has occurred in the below-knee region, and can be used for the treatment of the lesion 220 that has occurred in an other region such as an iliac artery.

First, the professional inserts a preceding guide wire into the blood vessel 200 (S110). Unlike the guide wire 100 of the present embodiment, the preceding guide wire is a known guide wire that does not have the leading portion 20. The preceding guide wire may be referred to as a workhorse guide wire, a first choice guide wire, or the like. The professional inserts the preceding guide wire into the blood vessel 200 via a sheath (not illustrated) disposed at a puncture position 230 (see FIG. 7) of the opposite leg 251. The professional advances the preceding guide wire to a location immediately proximal to the lesion 220 in the blood vessel 200 of the target leg 252.

Next, the professional inserts a catheter 120 into the blood vessel 200 along the preceding guide wire (S120). The professional advances the catheter 120 (see FIG. 7) to a location immediately proximal to the lesion 220 in the blood vessel 200.

Next, the professional removes the preceding guide wire from the blood vessel 200 (S130). Thereafter, the professional inserts the guide wire 100 into the catheter 120 inserted into the blood vessel 200, with the leading portion 20 at the front (S140, FIG. 7). The professional advances the guide wire 100 to a location immediately proximal to the lesion 220 in the blood vessel 200. When advancing the guide wire 100, the professional may or may not rotate the guide wire 100 around the center axis Ax.

Next, while rotating the guide wire 100, the professional advances the guide wire 100 toward the distal end side, thereby causing the leading portion 20 of the guide wire 100 to enter the lesion 220 (S150, FIG. 8). When the professional grips the proximal end portion of the guide wire 100 and rotates the guide wire 100 around the center axis Ax, the leading portion 20 located at the distal end portion of the guide wire 100 also rotates around the center axis Ax. The leading portion 20, which is rotating within the lesion 220, drills the lesion 220 in a manner that tears apart the lesion. In the present embodiment, in the step of advancing the guide wire 100 (S150), the professional advances the leading portion 20 until the leading portion 20 passes through the lesion 220. In a case where the leading portion 20 has an edge, when the leading portion 20 rotates around the center axis Ax in a state where the edge is in contact with the lesion 220, the edge scrapes the lesion 220. The leading portion 20 advances into a space formed by the edge scraping the lesion 220, thereby forming a through-hole in the lesion 220. As described above, the spiral groove 18 is formed on the outer peripheral surface 17 of the main body part 10. Due to the presence of the groove 18, the main body part 10 has a function of discharging small pieces of the lesion 220 generated by contact between the leading portion 20, which is rotating, and the lesion 220 from the distal end side toward the proximal end side of the main body part 10 (referred to as “lesion discharge function”). The step of advancing the guide wire 100 (S150) is performed in a state where no other medical devices have passed through the lesion 220.

After the leading portion 20 of the guide wire 100 has passed through the lesion 220, the professional advances a catheter (not illustrated) to the location of the lesion 220 along the guide wire 100. Thereafter, the professional removes the guide wire 100. When removing the guide wire 100, the professional may or may not rotate the guide wire 100 around the center axis Ax.

Thereafter, the professional inserts a guide wire for an adjunctive device (not illustrated) into the blood vessel 200 and advances the guide wire for an adjunctive device until the distal end of the guide wire for an adjunctive device passes through the lesion 220. The professional advances the adjunctive device to the location of the lesion 220 along the guide wire for the adjunctive device. The adjunctive device may be, for example, a device for atherectomy, a balloon catheter, a stent, and the like.

### Detailed Configuration of Guide Wire 100

As illustrated in FIG. 3, the guide wire 100 includes a wire 40 and a coil 50.

The coil 50 is a cylindrical member in which one or more wires are spirally wound. The main body part 10 includes the coil 50. An outer diameter of the coil 50 is, for example, not less than 0.1 mm and not more than 0.6 mm. The outer diameter of the coil 50 may be not less than 0.2 mm and not more than 0.5 mm, or may be not less than 0.3 mm and not more than 0.4 mm. The outer diameter of the coil 50 may be not less than 1.00 mm and not more than 2.00 mm, may be not less than 1.10 mm and not more than 1.65 mm, or may be not less than 1.20 mm and not more than 1.35 mm. In the present embodiment, the outer diameter of the coil 50 is constant over the entire length of the coil 50. The coil 50 may have a tapered shape in which the outer diameter of the coil 50 gradually decreases from a proximal end toward a distal end, or may have a tapered shape in which the outer diameter of the coil 50 gradually decreases from the distal end toward the proximal end. A spiral groove 53 is formed on an outer peripheral surface 52 of the coil 50. In the present embodiment, the coil 50 is Z-wound, and the running direction of the groove 53 is also a direction of Z-winding. Due to the presence of the spiral groove 53, the spiral groove 18 is formed on the outer peripheral surface 17 of the main body part 10. The distal end of the coil 50 coincides with the distal end 16 of the main body part 10.

The wire forming the coil 50 may be a single strand or a twisted wire formed by twisting a plurality of strands. In the present embodiment, the coil 50 is a multi-thread coil formed by winding a plurality of wires. In the present embodiment, each wire forming the coil 50 is a twisted wire.

As a material forming the coil 50, for example, a metal is used. More specifically, for example, radiolucent materials such as stainless steels (SUS302, SUS304, SUS316, etc.), Ni-Ti alloys, piano wire, and radiopaque materials such as platinum, gold, tungsten, and any of alloys thereof may be used. The coil 50 may be wholly formed of the same material, or individual portions may each be formed of different materials.

The wire 40 is a linear member. The wire 40 includes a base portion 40A, a folded portion 40B, and a loop portion 40C. In the wire 40, the base portion 40A, the loop portion 40C, and the folded portion 40B are arranged in this order from a first end 40E1 toward a second end 40E2 of the wire 40. The base portion 40A and the loop portion 40C are continuous in a longitudinal direction of the wire 40. The loop portion 40C and the folded portion 40B are continuous in the longitudinal direction of the wire 40. The main body part 10 includes the base portion 40A and the folded portion 40B of the wire 40. The leading portion 20 includes the loop portion 40C of the wire 40.

The base portion 40A is a portion of the wire 40. A proximal end of the base portion 40A is located at a proximal end portion of the main body part 10. The proximal end side of the base portion 40A is a portion to be gripped by the professional. A distal end of the base portion 40A is located at the distal end portion of the main body part 10.

The base portion 40A includes a large diameter portion 41, a first tapered portion 42, an intermediate diameter portion 43, a second tapered portion 44, and a small diameter portion 47. In the base portion 40A, the large diameter portion 41, the first tapered portion 42, the intermediate diameter portion 43, the second tapered portion 44, and the small diameter portion 47 are arranged in this order from the proximal end side toward the distal end side of the guide wire 100.

The large diameter portion 41 is a rod-shaped portion having a substantially constant outer diameter. The outer diameter (maximum width) of the large diameter portion 41 is, for example, about not less than 0.2 mm and not more than 3.0 mm. The first tapered portion 42 is a portion whose diameter gradually decreases from a boundary with the large diameter portion 41 toward a boundary with the intermediate diameter portion 43. The intermediate diameter portion 43 is a rod-shaped portion having a substantially constant outer diameter that is smaller than the outer diameter of the large diameter portion 41. The second tapered portion 44 is a portion whose diameter gradually decreases from the boundary with the intermediate diameter portion 43 toward a boundary with the small diameter portion 47. In the present embodiment, the second tapered portion 44 includes a proximal-end-side second tapered portion 45 and a distal-end-side second tapered portion 46 located on the distal end side with respect to the proximal-end-side second tapered portion 45. The small diameter portion 47 is a rod-shaped portion having a substantially constant outer diameter that is smaller than the outer diameter of the intermediate diameter portion 43.

In the present embodiment, rates of change (hereinafter, referred to as “gradients”) of the outer diameters along the longitudinal direction in the proximal-end-side second tapered portion 45 and the distal-end-side second tapered portion 46 are different from each other. For example, the gradient of the proximal-end-side second tapered portion 45 is steeper than the gradient of the distal-end-side second tapered portion 46. The gradient of the proximal-end-side second tapered portion 45 may be gentler than the gradient of the distal-end-side second tapered portion 46, or may be the same as the gradient of the distal-end-side second tapered portion 46. In the present embodiment, gradients of the first tapered portion 42 and the second tapered portion 44 are different from each other. For example, the gradient of the first tapered portion 42 is steeper than the gradient of the second tapered portion 44. The gradient of the first tapered portion 42 may be gentler than the gradient of the second tapered portion 44, or may be the same as the gradient of the second tapered portion 44.

The folded portion 40B is a portion of the wire 40 that is different from the base portion 40A. The folded portion 40B has a portion facing the base portion 40A in the Y-axis direction intersecting the axial direction of the main body part 10. A proximal end of the folded portion 40B is located on the distal end side with respect to the proximal end of the base portion 40A. A distal end of the folded portion 40B is located at the distal end portion of the main body part 10.

The loop portion 40C is a portion of the wire 40 that is different from the base portion 40A and the folded portion 40B. The loop portion 40C is located between the base portion 40A and the folded portion 40B in the longitudinal direction of the wire 40. The loop portion 40C has a loop shape when viewed in the X-axis direction. The loop portion 40C surrounds the through-hole 24 in the leading portion 20. An outer edge of the loop portion 40C in the present embodiment has a heart shape when viewed in the X-axis direction (see FIG. 4). In more detail, the loop portion 40C extends from a boundary position 40P1, which is a boundary position with the base portion 40A, toward a bending position 40P2 located on the distal end side and radially outward with respect to the boundary position 40P1. The loop portion 40C extends from the bending position 40P2 toward a bending position 40P3 located on the proximal end side and radially inward with respect to the bending position 40P2. The bending position 40P3 is located on the center axis Ax. The loop portion 40C extends from the bending position 40P3 toward a bending position 40P4 located on the distal end side with respect to the bending position 40P3 and on a side opposite to the bending position 40P2 across the center axis Ax. The loop portion 40C extends from the bending position 40P4 toward the proximal end side to a boundary position 40P5 that is a boundary position with the folded portion 40B and that is located on the proximal end side and radially inward with respect to the bending position 40P4. The boundary position 40P1 and the boundary position 40P5 are proximal ends of the loop portion 40C. The proximal ends of the loop portion 40C are located at a proximal end portion of the leading portion 20. The bending position 40P2 and the bending position 40P4 are distal ends of the loop portion 40C. The distal ends of the loop portion 40C are located at a distal end portion of the leading portion 20.

As a material forming the wire 40, for example, a metal is used. More specifically, for example, stainless steels (SUS302, SUS304, SUS316, etc.), Ni—Ti alloys, piano wire, and the like are used. The wire 40 may be wholly formed of the same material, or individual portions may each be formed of different materials.

The wire 40 is inserted into a hollow of the coil 50. The coil 50 covers at least a part of the distal end side of the base portion 40A and the folded portion 40B.

The coil 50 is joined to the wire 40 via a distal-end-side bonding material 61 formed at a distal end portion of the coil 50 and a proximal-end-side bonding material 62 formed at a proximal end portion of the coil 50. The base portion 40A and the folded portion 40B are connected to the coil 50 via the distal-end-side bonding material 61. The distal-end-side bonding material 61 protrudes from a distal end 51 of the coil 50 toward the distal end side. The loop portion 40C is connected to the coil 50 via a portion of the distal-end-side bonding material 61 protruding from the distal end 51 of the coil 50. The coil 50 may be joined to the wire 40 via a bonding material formed at an other position. As a material for forming the distal-end-side bonding material 61 and the proximal-end-side bonding material 62, for example, a metal solder (Au—Sn alloy, Sn—Ag alloy, Sn—Pb alloy, Pb—Ag alloy, etc.), a brazing material (aluminum alloy braze, silver braze, gold braze, etc.), an adhesive (epoxy-based adhesive, etc.), or the like is used.

The leading portion 20 has a reinforcing portion 28 located at a connection portion with the main body part 10. The connection portion of the leading portion 20 with the main body part 10 is reinforced by the reinforcing portion 28. In the present embodiment, the reinforcing portion 28 is formed of a portion of the distal-end-side bonding material 61 protruding from the distal end 51 of the coil 50 toward the distal end side. The reinforcing portion 28 may be formed by a welded portion between the leading portion 20 and the main body part 10. A constriction 29 is formed on an outer peripheral surface of the reinforcing portion 28. That is, in a part (for example, a proximal end portion) of the reinforcing portion 28, a width of the reinforcing portion 28 in a direction orthogonal to the axis Ax decreases toward a distal end of the reinforcing portion 28, and in an other part (for example, a distal end portion) of the reinforcing portion 28, a width of the reinforcing portion 28 in the direction orthogonal to the axis Ax increases toward the distal end of the reinforcing portion 28. Therefore, the outer peripheral surface of the reinforcing portion 28 has a curved surface that is recessed inward in a radial direction of the reinforcing portion 28.

As illustrated in FIG. 1, the guide wire 100 includes a first coating 31 and a second coating 32. The first coating 31 covers at least a part of the leading portion 20. The first coating 31 may cover a part of the main body part 10. In the present embodiment, the first coating 31 covers a region R31 formed by the entire leading portion 20 and the distal end portion of the main body part 10. The second coating 32 covers at least a part of the main body part 10. In the present embodiment, the second coating 32 covers a region R32 which is an intermediate portion of the main body part 10 excluding the distal end portion and the proximal end portion. The region R32 is located on a proximal end side of the region R31. A slidability of the first coating 31 is different from a slidability of the second coating 32. For example, the slidability of the first coating 31 is lower than the slidability of the second coating 32. The first coating 31 is, for example, hydrophobic and is formed of silicone. The second coating 32 is, for example, hydrophilic, and is formed of polyvinylpyrrolidone, polyacrylic acid, polyacrylamide, polyvinyl alcohol, a maleic anhydride copolymer, hyaluronic acid, or the like.

The guide wire 100 further includes a third coating 33. The third coating 33 covers at least a part of the main body part 10. In the present embodiment, the third coating 33 covers a region R33, which is the proximal end portion of the main body part 10. The region R33 is located on a proximal end side of the region R32. The third coating 33 is formed of, for example, PTFE.

As illustrated in FIG. 4, when viewed from the X-axis direction orthogonal to the axial direction of the main body part 10, an outer edge of the leading portion 20 satisfies the following first condition.

First condition: The outer edge of the leading portion 20 has a first end portion P1 located at the distal end 23 of the leading portion 20 and a second end portion P2 that is convex toward the distal end side and that is located on a side opposite to the first end portion P1 across the center axis Ax of the main body part 10 in the Y-axis direction orthogonal to both the axial direction of the main body part 10 and the X-axis direction.

In the present embodiment, the first end portion P1 coincides with the bending position 40P2 of the wire 40. In the present embodiment, the second end portion P2 coincides with the bending position 40P4 of the wire 40. As illustrated in FIG. 4, the leading portion 20 has a concave portion P3 between the first end portion P1 and the second end portion P2 in the Y-axis direction. The concave portion P3 is a portion recessed toward the proximal end side from each of the first end portion P1 and the second end portion P2. In present embodiment, the concave portion P3 coincides with the bending position 40P3 of the wire 40. The concave portion P3 is located on the center axis Ax.

In the present embodiment, a curvature at the first end portion P1 is equal to a curvature at the second end portion P2. In detail, the periphery of the first end portion P1 is curved as viewed in the X-axis direction. The periphery of the second end portion P2 is curved as viewed in the X-axis direction. A degree of bending of the loop portion 40C at the periphery of the first end portion P1 and a degree of bending of the loop portion 40C at the periphery of the second end portion P2 are equivalent to each other. The curvature at the first end portion P1 may be larger than the curvature at the second end portion P2, or may be smaller than the curvature at the second end portion P2.

In the present embodiment, in the axial direction of the main body part 10, a distance d1 from the distal end 16 of the main body part 10 to the first end portion P1 is equal to a distance d2 from the distal end 16 of the main body part 10 to the second end portion P2. In other words, the second end portion P2 is located at the distal end 23 of the leading portion 20. When the main body part 10 has a coil, the distal end 16 of the main body part 10 can be the distal end of the coil. In the present embodiment, a distance sd1 from the center axis Ax of the main body part 10 to the first end portion P1 in the Y-axis direction is equal to a distance sd2 from the center axis Ax of the main body part 10 to the second end portion P2 in the Y-axis direction. In the present embodiment, a distance d40 from the first end portion P1 to the second end portion P2 in the Y-axis direction is larger than a width w10 of the distal end portion of the main body part 10 in the Y-axis direction. A ratio of the distance d40 to the width w10, d40/w10, is, for example, not less than 1.1 and not more than 2.0. d40/w10 may be not less than 1.2 and not more than 1.8, or may be not less than 1.25 and not more than 1.5. The distance d40 may be equal to the width w10 or may be smaller than the width w10. In the present embodiment, in the Y-axis direction, each of the first end portion P1 and the second end portion P2 is farther away from the center axis Ax of the main body part 10 than end portions 16E1 and 16E2 of the distal end portion of the main body part 10 in the Y-axis direction. In the Y-axis direction, at least one of the first end portion P1 and the second end portion P2 may be closer to the center axis Ax of the main body part 10 than the end portions 16E1 and 16E2 of the distal end portion of the main body part 10 in the Y-axis direction.

### Method of Manufacturing Guide Wire 100

The guide wire 100 according to the present embodiment can be manufactured, for example, by the following manufacturing method. First, a worker prepares the wire 40. Next, the worker inserts the wire 40 into a hollow portion of the coil 50. At this time, the worker arranges the wire 40 in the hollow portion of the coil 50 so that a part of the wire 40 on the distal end side protrudes from the distal end of the coil 50. Next, the worker bends the portion of the wire 40 protruding from the distal end of the coil 50 into a loop shape. As a result, portions corresponding to the base portion 40A, the folded portion 40B, and the loop portion 40C of the wire 40 are formed. Next, the worker inserts the portion corresponding to the folded portion 40B of the wire 40 into the hollow portion of the coil 50.

Next, the worker joins the wire 40 and the coil 50. Specifically, to join the wire 40 to the coil 50, the worker forms the distal-end-side bonding material 61 and the proximal-end-side bonding material 62 with a bonding material such as a solder material. The bonding material is supplied, for example, between the strands of the coil 50 to the inside of the coil 50. The reinforcing portion 28 is formed by the distal-end-side bonding material 61 protruding from the distal end of the coil 50 toward the distal end side. By the joining step, the guide wire 100 including the leading portion 20 having the reinforcing portion 28 and the main body part 10 is manufactured.

### Effect of Present Embodiment

As described above, the guide wire 100 according to the present embodiment includes the elongated main body part 10 and the leading portion 20 that is connected to the distal end 16 of the main body part 10 and enters the lesion 220. When viewed from the X-axis direction orthogonal to the axial direction of the main body part 10, the outer edge of the leading portion 20 satisfies the first condition that the outer edge has the first end portion P1 located at the distal end 23 of the leading portion 20 and the second end portion P2 located on a side opposite to the first end portion P1 across the center axis Ax of the main body part 10 in the Y-axis direction orthogonal to both the axial direction of the main body part 10 and the X-axis direction, the second end portion P2 being convex toward the distal end side. According to the guide wire 100 of the present embodiment, since the outer edge of the leading portion 20 has the first end portion P1 and the second end portion P2, the leading portion 20 is easily caught by the lesion 220, and the passability of the guide wire 100 through the lesion 220 is improved.

In the guide wire 100 of present embodiment, the curvature at the first end portion P1 is equal to the curvature at the second end portion P2. According to the guide wire 100 of the present embodiment, the leading portion 20 is easily caught by the lesion 220, and the passability of the guide wire 100 through the lesion 220 is improved.

In the guide wire 100 of the present embodiment, in the axial direction of the main body part 10, the distance d1 from the distal end 16 of the main body part 10 to the first end portion P1 is equal to the distance d2 from the distal end 16 of the main body part 10 to the second end portion P2. According to the guide wire 100 of the present embodiment, the leading portion 20 is easily caught by the lesion 220, and the passability of the guide wire 100 through the lesion 220 is improved.

In the guide wire 100 of the present embodiment, the distance sd1 from the center axis Ax of the main body part 10 to the first end portion P1 in the Y-axis direction is equal to the distance sd2 from the center axis Ax of the main body part 10 to the second end portion P2 in the Y-axis direction. According to the guide wire 100 of the present embodiment, the leading portion 20 is easily caught by the lesion 220, and the passability of the guide wire 100 through the lesion 220 is improved.

In the guide wire 100 of the present embodiment, the distance d40 from the first end portion P1 to the second end portion P2 in the Y-axis direction is larger than the width w10 of the distal end portion of the main body part 10 in the Y-axis direction. According to the guide wire 100 of the present embodiment, the leading portion 20 can efficiently drill the lesion 220, and the passability of the guide wire 100 through the lesion 220 is improved.

In the guide wire 100 of the present embodiment, in the Y-axis direction, each of the first end portion P1 and the second end portion P2 is farther away from the center axis Ax of the main body part 10 than the end portions 16E1 and 16E2 of the distal end portion of the main body part 10 in the Y-axis direction. According to the guide wire 100 of the present embodiment, the leading portion 20 can efficiently drill the lesion 220, and the passability of the guide wire 100 through the lesion 220 is improved.

### B. Second Embodiment

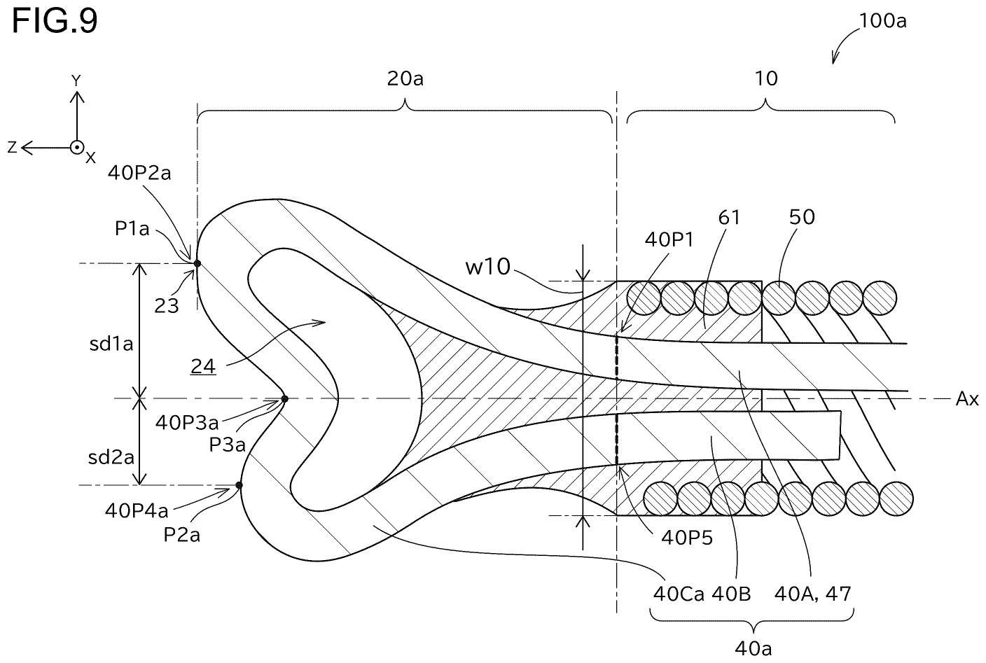

FIG. 9 is an explanatory view schematically illustrating a configuration of a guide wire 100a according to a second embodiment. FIG. 9 illustrates a YZ longitudinal section of a distal end portion of the guide wire 100a. Hereinafter, components of the guide wire 100a according to the second embodiment, which are the same as the components of the guide wire 100 according to the first embodiment, are denoted with the same reference signs, thereby omitting the description thereof as appropriate.

In the guide wire 100a according to the second embodiment, an aspect of a leading portion 20a is different from the aspect of the leading portion 20 in the guide wire 100 according to the first embodiment. In detail, the leading portion 20a includes a loop portion 40Ca. The loop portion 40Ca extends from a boundary position 40P1 toward a bending position 40P2a located on a distal end side and radially outward with respect to the boundary position 40P1. The loop portion 40Ca extends from the bending position 40P2a toward a bending position 40P3a located on a proximal end side and radially inward with respect to the bending position 40P2a. The loop portion 40Ca extends from the bending position 40P3a toward a bending position 40P4a located on the distal end side with respect to the bending position 40P3a and on a side opposite to the bending position 40P2a across a center axis Ax. The loop portion 40Ca extends from the bending position 40P4a toward the proximal end side to a boundary position 40P5 located on the proximal end side and radially inward with respect to the bending position 40P4a. The bending position 40P2a is a distal end of the loop portion 40Ca. The bending position 40P4a is located on the proximal end side with respect to the bending position 40P2a.

When viewed from an X-axis direction, an outer edge of the leading portion 20a satisfies the first condition, similarly to the outer edge of the leading portion 20 in the first embodiment. In the present embodiment, a first end portion P1a coincides with the bending position 40P2a of a wire 40a. In the present embodiment, a second end portion P2a coincides with the bending position 40P4a of the wire 40a. In present embodiment, a concave portion P3a coincides with the bending position 40P3a of the wire 40a.

In the present embodiment, a curvature at the first end portion P1a is smaller than a curvature at the second end portion P2a. In detail, a degree of bending of the loop portion 40Ca at the periphery of the first end portion P1a is gentler than a degree of bending of the loop portion 40Ca at the periphery of the second end portion P2a. The curvature at the first end portion P1a may be larger than the curvature at the second end portion P2a, or may be equal to the curvature at the second end portion P2a.

In the present embodiment, the second end portion P2a is located on the proximal end side with respect to the first end portion P1a in the axial direction of a main body part 10. In the present embodiment, a distance sd1a from the center axis Ax of the main body part 10 to the first end portion P1a in a Y-axis direction is different from a distance sd2a from the center axis Ax of the main body part 10 to the second end portion P2a in the Y-axis direction. In detail, the distance sd1a is greater than the distance sd2a. The distance sd1a may be shorter than the distance sd2a.

As described above, in the guide wire 100a of the present embodiment, the outer edge of the leading portion 20a satisfies the first condition, and the curvature at the first end portion P1a is smaller than the curvature at the second end portion P2a. According to the guide wire 100a of the present embodiment, since the first end portion P1a having a relatively small curvature can easily drill a lesion 220, the passability of the guide wire 100a through the lesion 220 is improved.

In the guide wire 100a of the present embodiment, the second end portion P2a is located on the proximal end side with respect to the first end portion P1a. According to the guide wire 100a of the present embodiment, the leading portion 20a is easily caught by the lesion 220, and the passability of the guide wire 100a through the lesion 220 is improved.

In the guide wire 100a of the present embodiment, the distance sd1a from the center axis Ax of the main body part 10 to the first end portion P1a in the Y-axis direction is different from the distance sd2a from the center axis Ax of the main body part 10 to the second end portion P2a in the Y-axis direction. According to the guide wire 100a of the present embodiment, the leading portion 20a is easily caught by the lesion 220, and the passability of the guide wire 100a through the lesion 220 is improved.

### C. Third Embodiment

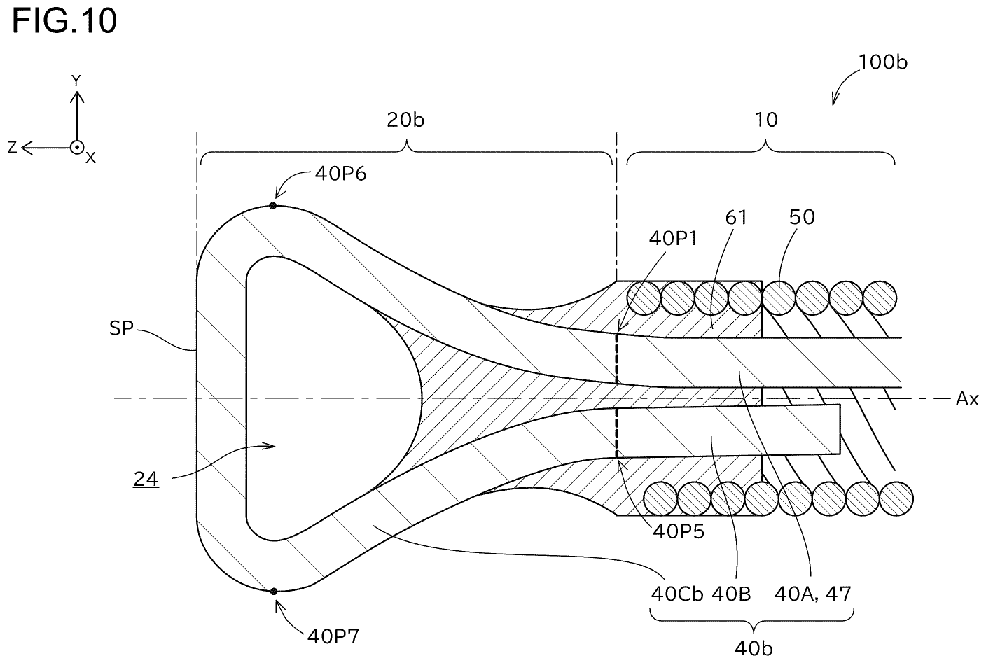

FIG. 10 is an explanatory view schematically illustrating a configuration of a guide wire 100b according to a third embodiment. FIG. 10 illustrates a YZ longitudinal section of a distal end portion of the guide wire 100b. Hereinafter, components of the guide wire 100b according to the third embodiment, which are the same as the components of the guide wire 100 according to the first embodiment, are denoted with the same reference signs, thereby omitting the description thereof as appropriate.

In the guide wire 100b according to the third embodiment, an aspect of a leading portion 20b is different from the aspect of the leading portion 20 in the guide wire 100 according to the first embodiment. In detail, the leading portion 20b includes a loop portion 40Cb. An outer edge of the loop portion 40Cb in the present embodiment is substantially triangular. In more detail, the loop portion 40Cb extends from a boundary position 40P1 toward an end portion 40P6 located on a distal end side and radially outward with respect to the boundary position 40P1. The loop portion 40Cb linearly extends in parallel to a Y-axis direction from the end portion 40P6 toward an end portion 40P7 located on a side opposite to the end portion 40P6 across a center axis Ax. The loop portion 40Cb extends from the end portion 40P7 toward a proximal end side to a boundary position 40P5 located on the proximal end side and radially inward with respect to the end portion 40P7.

As illustrated in FIG. 10, when viewed from an X-axis direction orthogonal to an axial direction of a main body part 10, an outer edge of the leading portion 20b satisfies the following second condition.

Second condition: The outer edge has a straight portion SP extending in the Y-axis direction.

In the present embodiment, the straight portion SP coincides with a portion of the loop portion 40Cb extending from the end portion 40P6 toward the end portion 40P7. In the present embodiment, the straight portion SP is parallel to the Y-axis direction. In other words, the straight portion SP is orthogonal to the center axis Ax of the main body part 10. In the present embodiment, the straight portion SP is oriented toward the distal end side of the guide wire 100b. The “straight portion” in the present specification includes not only a strictly linear portion but also a shape slightly deviated from a linear shape due to inevitable circumstances such as a manufacturing error.

As described above, the guide wire 100b of the present embodiment includes the elongated main body part 10 and the leading portion 20b that is connected to a distal end 16 of the main body part 10 and enters a lesion 220. When viewed from the X-axis direction orthogonal to the axial direction of the main body part 10, the outer edge of the leading portion 20 satisfies the second condition that the outer edge of the leading portion 20b has the straight portion SP extending in the Y-axis direction. According to the guide wire 100b of the present embodiment, since the outer edge of the leading portion 20b has the straight portion SP, the leading portion 20b is easily caught by the lesion 220, and the passability of the guide wire 100b through the lesion 220 is improved.

In the guide wire 100b of the present embodiment, the straight portion SP is orthogonal to the center axis Ax of the main body part 10. According to the guide wire 100b of the present embodiment, the leading portion 20b is easily caught by the lesion 220, and the passability of the guide wire 100b through the lesion 220 is improved.

### D. Fourth Embodiment

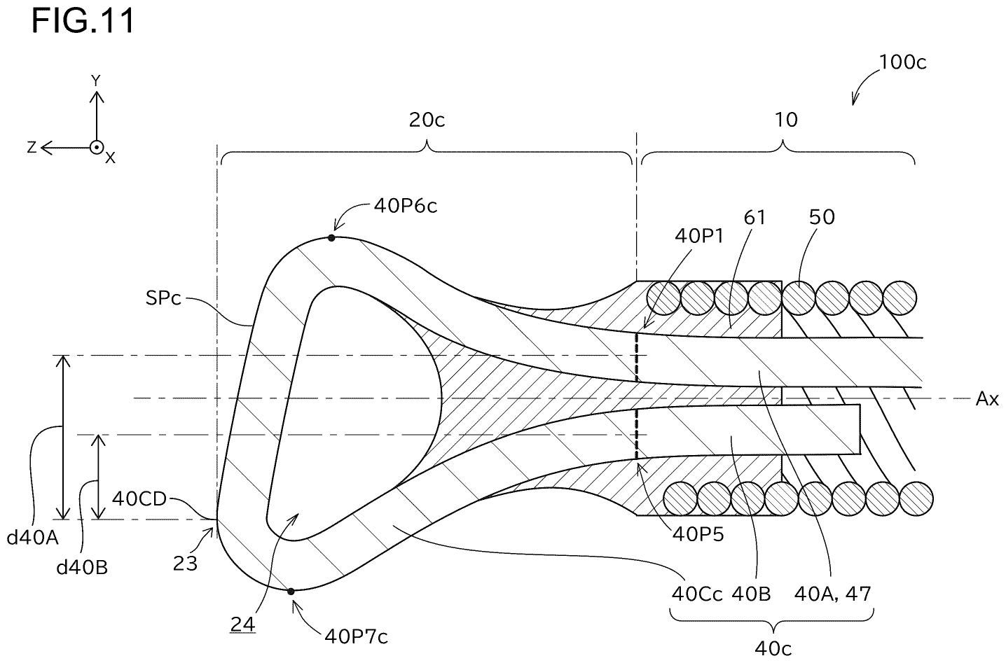

FIG. 11 is an explanatory view schematically illustrating a configuration of a guide wire 100c according to a fourth embodiment. FIG. 11 illustrates a YZ longitudinal section of a distal end portion of the guide wire 100c. Hereinafter, components of the guide wire 100c according to the fourth embodiment, which are the same as the components of the guide wire 100b according to the third embodiment, are denoted with the same reference signs, thereby omitting the description thereof as appropriate.

In the guide wire 100c according to the fourth embodiment, an aspect of a leading portion 20c is different from the aspect of the leading portion 20b in the guide wire 100b of the third embodiment. In detail, the leading portion 20c includes a loop portion 40Cc of a wire 40c. An outer edge of the loop portion 40Cc in the present embodiment is substantially triangular. The loop portion 40Cc extends from a boundary position 40P1 toward an end portion 40P6c located on a distal end side and radially outward with respect to the boundary position 40P1. The loop portion 40Cc linearly extends from the end portion 40P6c toward an end portion 40P7c located on the distal end side with respect to the end portion 40P6c and on a side opposite to the end portion 40P6c across a center axis Ax. The loop portion 40Cc extends from the end portion 40P7c toward a proximal end side to a boundary position 40P5 located on the proximal end side and radially inward with respect to the end portion 40P7c.

An outer edge of the leading portion 20c in the fourth embodiment satisfies the second condition, similarly to the outer edge of the leading portion 20b in the third embodiment. In the present embodiment, a straight portion SPc coincides with a portion of the loop portion 40Cc extending from the end portion 40P6c toward the end portion 40P7c. In the present embodiment, the straight portion SPc extends in a Y-axis direction while having a predetermined angle with respect to the Y-axis direction orthogonal to the axial direction of a main body part 10. The predetermined angle is, for example, not more than 45°. The predetermined angle may be not more than 30°, or may be not more than 15°. As in the present embodiment, the straight portion may have a predetermined angle with respect to the Y-axis direction.

The loop portion 40Cc has a distal end part 40CD located at a distal end 23 of the leading portion 20c. In the present embodiment, the distal end part 40CD is located at an end portion of the straight portion SPc in the Y-axis direction. As illustrated in FIG. 11, a distance d40B between a position of the distal end part 40CD and a position of a folded portion 40B in the Y-axis direction is shorter than a distance d40A between the position of the distal end part 40CD and a position of a base portion 40A in the Y-axis direction. In other words, in the Y-axis direction, the base portion 40A and the folded portion 40B are located on opposite sides to each other across the center axis Ax. The distal end part 40CD is located on the same side as the folded portion 40B with respect to the center axis Ax.

As described above, in the guide wire 100c of the present embodiment, the outer edge of the leading portion 20c satisfies the second condition, and the straight portion SPc extends in the Y-axis direction while having a predetermined angle with respect to the Y-axis direction. According to the guide wire 100c of the present embodiment, the leading portion 20c is easily caught by a lesion 220, and the passability of the guide wire 100c through the lesion 220 is improved.

In the guide wire 100c of the present embodiment, the main body part 10 includes the base portion 40A that is a portion of the wire 40c, and the folded portion 40B that is a portion of the wire 40c different from the base portion 40A and whose proximal end is located on the distal end side with respect to a proximal end of the base portion 40A. The leading portion 20c includes the loop portion 40Cc that is a portion of the wire 40c different from the base portion 40A and the folded portion 40B and is located between the base portion 40A and the folded portion 40B of the wire 40c. The loop portion 40Cc has the distal end part 40CD located at a distal end 23 of the leading portion 20c. The distance d40B between the position of the distal end part 40CD and the position of the folded portion 40B in the Y-axis direction is shorter than the distance d40A between the position of the distal end part 40CD and the position of the base portion 40A in the Y-axis direction. According to the guide wire 100c of the present embodiment, the leading portion 20c is easily caught by the lesion 220, and the passability of the guide wire 100c through the lesion 220 is improved.

### E. Fifth Embodiment

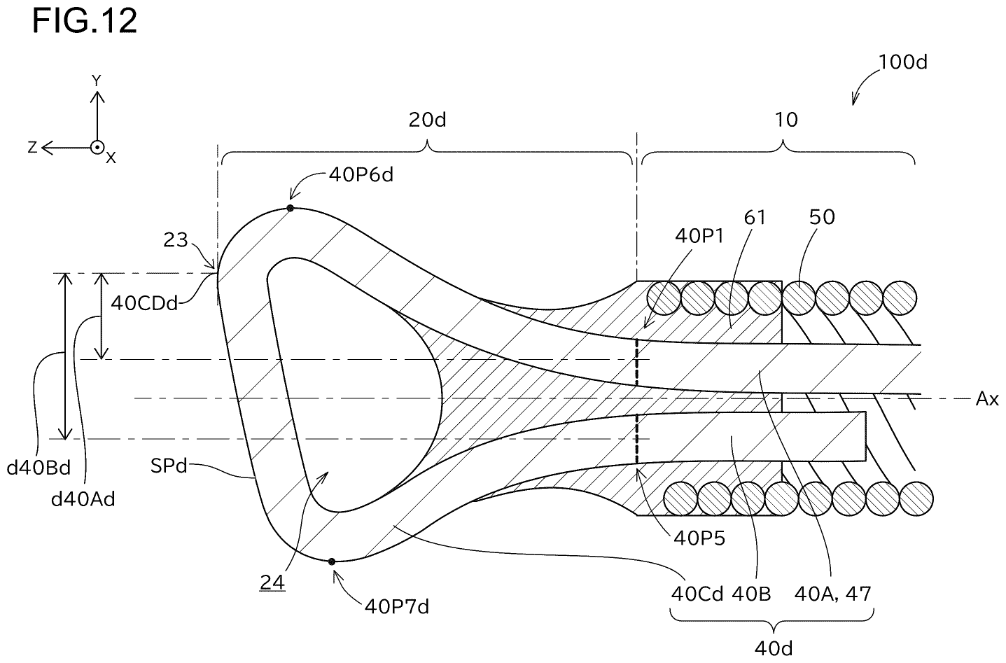

FIG. 12 is an explanatory view schematically illustrating a configuration of a guide wire 100d according to a fifth embodiment. FIG. 12 illustrates a YZ longitudinal section of a distal end portion of the guide wire 100d. Hereinafter, components of the guide wire 100d according to the fifth embodiment, which are the same as the components of the guide wire 100b according to the third embodiment, are denoted with the same reference signs, thereby omitting the description thereof as appropriate.

In the guide wire 100d according to the fifth embodiment, an aspect of a leading portion 20d is different from the aspect of the leading portion 20b in the guide wire 100b according to the third embodiment. In detail, the leading portion 20d includes a loop portion 40Cd of a wire 40d. An outer edge of the loop portion 40Cd in the present embodiment is substantially triangular. The loop portion 40Cd extends from a boundary position 40P1 toward an end portion 40P6d located on a distal end side and radially outward with respect to the boundary position 40P1. The loop portion 40Cd linearly extends from the end portion 40P6d toward an end portion 40P7d located on a proximal end side with respect to the end portion 40P6d and on a side opposite to the end portion 40P6d across a center axis Ax. The loop portion 40Cd extends from the end portion 40P7d toward the proximal end side to a boundary position 40P5 located on the proximal end side and radially inward with respect to the end portion 40P7d.

An outer edge of the leading portion 20d in the fifth embodiment satisfies the second condition, similarly to the outer edge of the leading portion 20b in the third embodiment. In the present embodiment, a straight portion SPd coincides with a portion of the loop portion 40Cd extending from the end portion 40P6d toward the end portion 40P7d. In the present embodiment, the straight portion SPd extends in a Y-axis direction while having a predetermined angle with respect to the Y-axis direction orthogonal to an axial direction of a main body part 10. The predetermined angle is, for example, not more than 45°. The predetermined angle may be not more than 30°, or may be not more than 15°. As in the present embodiment, the straight portion may have a predetermined angle with respect to the Y-axis direction.

The loop portion 40Cd has a distal end part 40CDd located at a distal end 23 of the leading portion 20d. In the present embodiment, the distal end part 40CDd is located at an end portion of the straight portion SPd in the Y-axis direction. As illustrated in FIG. 12, a distance d40Ad between a position of the distal end part 40CDd and a position of a base portion 40A in the Y-axis direction is shorter than a distance d40Bd between the position of the distal end part 40CDd and a position of a folded portion 40B in the Y-axis direction. In other words, in the Y-axis direction, the base portion 40A and the folded portion 40B are located on opposite sides to each other across the center axis Ax. The distal end part 40CDd is located on the same side as the base portion 40A with respect to the center axis Ax.

As described above, in the guide wire 100d of the present embodiment, the main body part 10 includes the base portion 40A that is a portion of the wire 40d, and the folded portion 40B that is a portion of the wire 40d different from the base portion 40A and whose proximal end is located on the distal end side with respect to a proximal end of the base portion 40A. The leading portion 20d includes the loop portion 40Cd that is a portion of the wire 40d that is different from the base portion 40A and the folded portion 40B and is located between the base portion 40A and the folded portion 40B of the wire 40d. The loop portion 40Cd has the distal end part 40CDd located at a distal end 23 of the leading portion 20d. The distance d40Ad between the position of the distal end part 40CDd and the position of the base portion 40A in the Y-axis direction is shorter than the distance d40Bd between the position of the distal end part 40CDd and the position of the folded portion 40B in the Y-axis direction. According to the guide wire 100d of the present embodiment, the leading portion 20d is easily caught by a lesion 220, and the passability of the guide wire 100d through the lesion 220 is improved.

### F. Sixth Embodiment

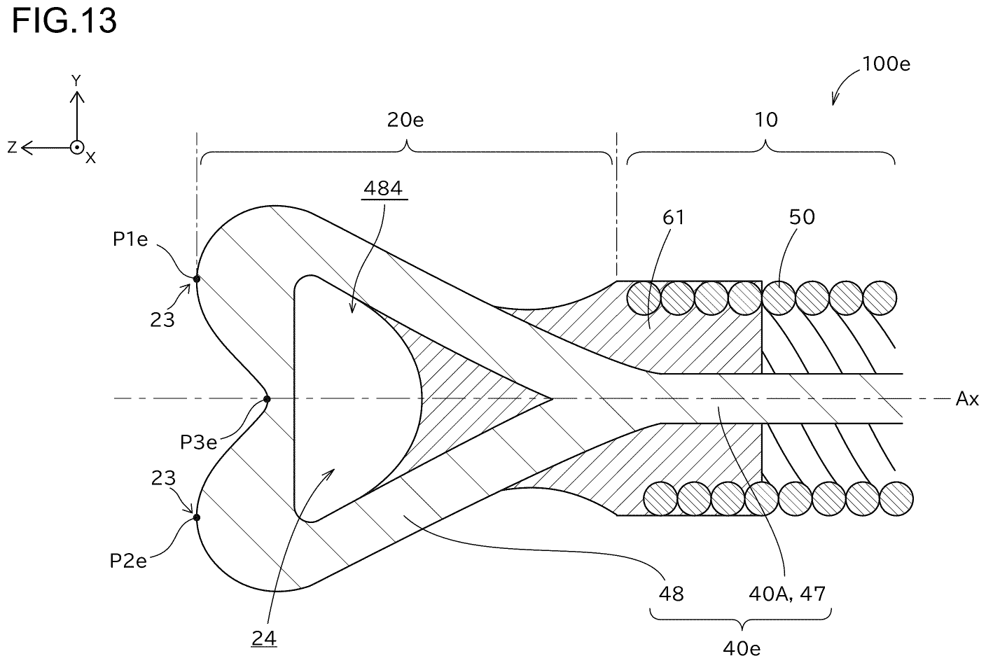

FIG. 13 is an explanatory view schematically illustrating a configuration of a guide wire 100e according to a sixth embodiment. FIG. 13 illustrates a YZ longitudinal section of a distal end portion of the guide wire 100e. Hereinafter, components of the guide wire 100e according to the sixth embodiment, which are the same as the components of the guide wire 100 according to the first embodiment, are denoted with the same reference signs, thereby omitting the description thereof as appropriate.

In the guide wire 100e according to the sixth embodiment, an aspect of a wire 40e is different from the aspect of the wire 40 in the first embodiment. The wire 40e includes a loop portion 48 continuous with a distal end of a base portion 40A instead of including the folded portion 40B and the loop portion 40C in the first embodiment. A through-hole 484 passing through the wire 40e in an X-axis direction is formed in the loop portion 48. In other words, the loop portion 48 has a loop shape surrounding the through-hole 484 as viewed in the X-axis direction. An outer edge of the loop portion 48 has a heart shape, similarly to the outer edge of the loop portion 40C in the first embodiment. Therefore, a leading portion 20e has a first end portion P1e, a second end portion P2e, and a concave portion P3e. The outer edge of the through-hole 484 is substantially triangular as viewed in the X-axis direction. The shape of the outer edge of the loop portion 48 and the shape of the outer edge of the through-hole 484 are not similar to each other. As in the present embodiment, the first end portion and the second end portion may not necessarily coincide with a folding-back position of the wire. As in the present embodiment, the outer edge of the loop portion and the outer edge of the through-hole may not be similar to each other.

### G. Seventh Embodiment

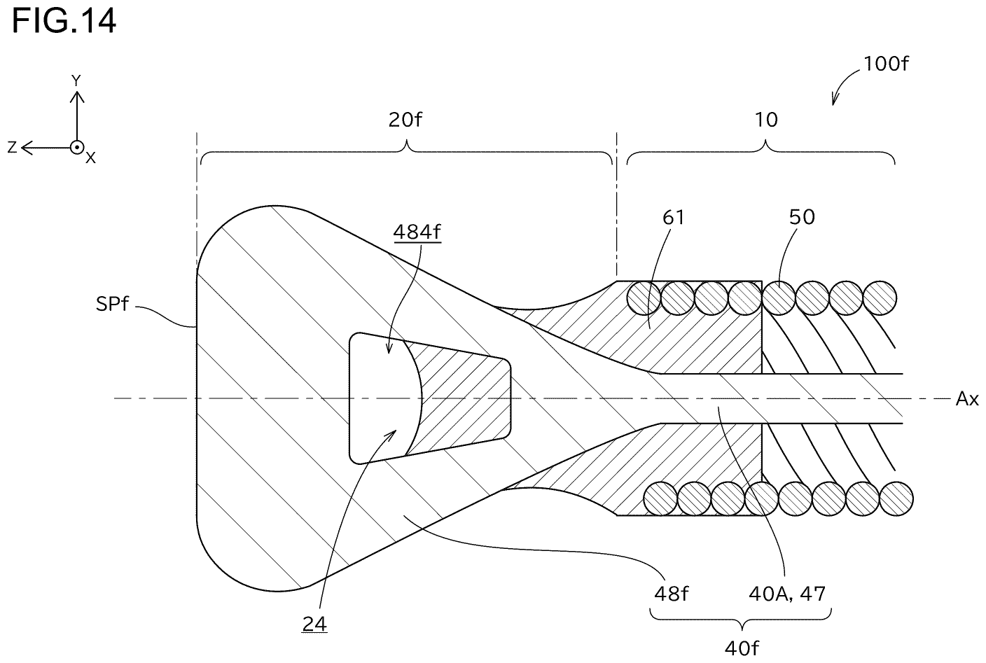

FIG. 14 is an explanatory view schematically illustrating a configuration of a guide wire 100f according to a seventh embodiment. FIG. 14 illustrates a YZ longitudinal section of a distal end portion of the guide wire 100f. Hereinafter, components of the guide wire 100f according to the seventh embodiment, which are the same as the components of the guide wire 100e according to the sixth embodiment, are denoted with the same reference signs, thereby omitting the description thereof as appropriate.

In the guide wire 100f according to the seventh embodiment, a configuration of a loop portion 48f in a wire 40f is different from the aspect of the loop portion 48 in the sixth embodiment. An outer edge of the loop portion 48f is substantially triangular, similarly to the outer edge of the loop portion 40Cc in the third embodiment. Therefore, a leading portion 20f has a straight portion SPf. An outer edge of the through-hole 484f is substantially quadrangular as viewed in an X-axis direction. The shape of the outer edge of the loop portion 48f and the shape of the outer edge of the through-hole 484f are not similar to each other. As in the present embodiment, the straight portion may not necessarily be a portion of the wire extending linearly. As in the present embodiment, the outer edge of the loop portion and the outer edge of the through-hole may not be similar to each other.

### H. Modifications

The technology disclosed in the present specification is not limited to the embodiments described above, and can be modified into various forms without departing from the gist thereof, and, for example, the following modifications can also be made.

The configurations of the guide wire 100 according to the above-described embodiments are merely examples and can be modified in various ways. For example, the guide wire may not include at least one of the first coating 31, the second coating 32, and the third coating 33.

The leading portion may not necessarily have a through-hole. That is, the leading portion may not necessarily have a loop shape.

The materials of the members in the above-described embodiments are merely examples and can be modified in various ways. The method of manufacturing the guide wire 100 in the above-described embodiment is merely an example and can be modified in various ways. The treatment method using the guide wire 100 in the above-described embodiment is merely an example, and can be modified in various ways.

In the above-described embodiments, the guide wire 100 for treating a lesion in a blood vessel has been described as an example. The technology disclosed herein is similarly applicable to general medical devices for treating a lesion in a living body lumen.

Each of all the features described in each of the embodiments described above may be appropriately combined with an other embodiment, or may be appropriately combined with the modification. Each of all the features described in each of the modifications described above may be appropriately combined with the embodiment or may be appropriately combined with an other modification. Each of all the features described in each of the embodiments described above may be omitted as appropriate. Each of all the features described in each of the modifications described above may be omitted as appropriate.

1. A medical device, comprising:
an elongated main body part; and
a leading portion connected to a distal end of the main body part, the leading portion entering a lesion, wherein,
when viewed from a first direction orthogonal to an axial direction of the main body part, an outer edge of the leading portion satisfies at least one of
a first condition that the outer edge has a first end portion located at a distal end of the leading portion and a second end portion located on a side opposite to the first end portion across a center axis of the main body part in a second direction orthogonal to both the axial direction of the main body part and the first direction, the second end portion being convex toward a distal end side, and
a second condition that the outer edge has a straight portion extending in the second direction.

2. The medical device according to claim 1, wherein
the outer edge of the leading portion satisfies the first condition, and
a curvature at the first end portion is equal to a curvature at the second end portion.

3. The medical device according to claim 1, wherein
the outer edge of the leading portion satisfies the first condition, and
a curvature at the first end portion is smaller than a curvature at the second end portion.

4. The medical device according to claim 1, wherein
the outer edge of the leading portion satisfies the first condition, and
in the axial direction of the main body part, a distance from the distal end of the main body part to the first end portion is equal to a distance from the distal end of the main body part to the second end portion.

5. The medical device according to claim 1, wherein
the outer edge of the leading portion satisfies the first condition, and
the second end portion is located on a proximal end side with respect to the first end portion.

6. The medical device according to claim 1, wherein
the outer edge of the leading portion satisfies the first condition, and
a distance from the center axis of the main body part to the first end portion in the second direction is equal to a distance from the center axis of the main body part to the second end portion in the second direction.

7. The medical device according to claim 1, wherein
the outer edge of the leading portion satisfies the first condition, and
a distance from the center axis of the main body part to the first end portion in the second direction is different from a distance from the center axis of the main body part to the second end portion in the second direction.

8. The medical device according to claim 1, wherein
the outer edge of the leading portion satisfies the first condition, and
a distance from the first end portion to the second end portion in the second direction is larger than a width of a distal end portion of the main body part in the second direction.

9. The medical device according to claim 1, wherein
the outer edge of the leading portion satisfies the first condition, and
in the second direction, each of the first end portion and the second end portion is farther away from the center axis of the main body part than an end portion of a distal end portion of the main body part in the second direction.

10. The medical device according to claim 1, wherein
the outer edge of the leading portion satisfies the second condition, and
the straight portion is orthogonal to the center axis of the main body part.

11. The medical device according to claim 1, wherein
the outer edge of the leading portion satisfies the second condition, and
the straight portion extends in the second direction while having a predetermined angle with respect to the second direction.

12. The medical device according to claim 1, wherein
the outer edge of the leading portion satisfies the second condition, and
the main body part includes
a base portion that is a portion of a wire and
a folded portion that is a portion of the wire different from the base portion, the folded portion having a proximal end located on a distal end side with respect to a proximal end of the base portion,
the leading portion includes a loop portion that is a portion of the wire different from the base portion and the folded portion and is located between the base portion and the folded portion of the wire,
the loop portion has a distal end part located at a distal end of the leading portion, and
a distance between a position of the distal end part and a position of the folded portion in the second direction is shorter than a distance between a position of the distal end part and a position of the base portion in the second direction.

13. The medical device according to claim 1, wherein
the outer edge of the leading portion satisfies the second condition, and
the main body part includes
a base portion that is a portion of a wire and
a folded portion that is a portion of the wire different from the base portion, the folded portion having a proximal end located on a distal end side with respect to a proximal end of the base portion,
the leading portion includes a loop portion that is a portion of the wire different from the base portion and the folded portion and is located between the base portion and the folded portion of the wire,
the loop portion has a distal end part located at a distal end of the leading portion, and
a distance between a position of the distal end part and a position of the base portion in the second direction is shorter than a distance between a position of the distal end part and a position of the folded portion in the second direction.
